(19) United States
(12) Patent Application Publication
(10) Pub. No.: US 2015/0335281 A1
(43) Pub. Date: Nov. 26, 2015

(54) MEDICAL DIAGNOSTIC TOOL BASED UPON NON-DESTRUCTIVE DETERMINATION OF THE MATERIAL COMPOSITION OF INTERNAL ORGANS AND TISSUES

(71) Applicant: C. Michael Scroggins, Richmond, TX (US)

(72) Inventor: C. Michael Scroggins, Richmond, TX (US)

(73) Assignee: C. Michael Scroggins, Richmond, TX (US)

(21) Appl. No.: 14/284,377

(22) Filed: May 21, 2014

(51) Int. Cl. A61B 5/00; A61B 5/055

(52) U.S. Cl. CPC A61B 5/4869; A61B 5/055; A61B 5/7246; A61B 5/7264

(57) ABSTRACT

Disclosed herein are technologies related to a tool for medical diagnostics based upon material composition of internal organs and tissues. This Abstract is submitted with the understanding that it will not be used to interpret or limit the scope or meaning of the claims.

### BACKGROUND

In general, the term “medical diagnosis” or “medical diagnostics” refers to the process of attempting to determine or identify a possible disease or disorder and/or to the opinion reached by this process. The process of medical diagnosis begins with medical information about an individual patient and culminates in categorizations regarding condition, prognosis, and treatment.

A medical diagnosis (or simply “diagnosis” herein), in the sense of diagnostic procedure, can be regarded as an attempt at classification of an individual's condition into separate and distinct categories that allow medical decisions about treatment and prognosis to be made. Subsequently, a diagnostic opinion is often described in terms of a disease or other condition.

A medical diagnosis typically begins with collection of data from and about a human (i.e., subject, patient, etc.). For example, medical staff takes the patients vital signs. For example, vital signs include, but are not limited, to body temperature, pulse rate (or heart rate), blood pressure, and respiratory rate. The patient may provide specific information about their health conditions (e.g., pain, sleep, diet, etc.). Monitoring devices (such as a pedometer, blood glucose meter, pulse/oximeter and thermometer) may provide other useful information.

In addition, the medical staff may collect other data about the patient in the form of medical radiology (i.e., medical imaging), which generates images of the human body (or parts and function thereof) for medical diagnostic purposes. Examples of medical radiological technologies include X-ray radiography, magnetic resonance imaging (MRI), medical ultrasonography or ultrasound, endoscopy, elastography, tactile imaging, thermography, medical photography and nuclear medicine functional imaging techniques as positron emission tomography.

Other measurement and recording techniques that are not primarily designed to produce images are considered part of medical radiology. Examples of such includes electroencephalography (EEG), magnetoencephalography (MEG), electrocardiography (EKG), and others, but which produce data susceptible to be represented as a parameter graph vs. time or maps which contain information about the measurement locations, can be considered as forms of medical imaging in a limited sense. Of course, still other monitoring devices (such as pedometer or blood glucose meter) may gather medical data about a patient.

Each of the devices discussed thus far is set to measure only specific singularities. Typically, a medical diagnosis based upon such measurements projects out from the data measurements in a bell-curve manner with upper and lower control limits. This is one reason that medical fields overlap findings and each field will apply their therapies (e.g., medications) for the organ/organs or fields in which the medical specialists specialize.

With regard to magnetic resonance imaging (MRI) in particular, it is based upon based on a nuclear magnetic resonance phenomenon where “free” hydrogen atoms (mainly from water) become field-parallel in a strong outer magnetic field. These hydrogen atoms reach a higher energy level by application of an additional electromagnetic field. After shutdown of this outer field the atoms send out electromagnetic Frequency waves (i.e., radio waves), representing the magnetic Frequency resonance signal.

With conventional approaches of use of an MRI, different amounts of “free” water result in various image characteristics. Typically, higher tissue water content is represented by high frequency magnetic resonance signals. Waterless structures show low frequency signals. These Frequency signals can be broken down into singularities for free Hydrogen atoms to show the potential combinations moving up the frequency scale to determine the three dimensional size and rate of change possible in cellular structures.

A diagnostic radiologist is a physician who reads and interprets digital images, such as those obtained via an MRI scan. The radiologist uses this information to help diagnose the patient and consult with the treating physician to develop a course of treatment. The radiologist's opinion is based upon an interpretation of the available medical imaging of the patient. That opinion can only be based upon what the radiologist sees in the medical image.

### BRIEF DESCRIPTION OF THE DRAWINGS

FIG. 1 shows an overview of an example operation in accordance with one or more implementations described herein.

FIG. 2 illustrates an example system in accordance with one or more implementations described herein.

FIG. 3 illustrates an example process in accordance with one or more implementations described herein.

FIG. 4 illustrates an example computing device to implement in accordance with the technologies described herein.

The Detailed Description references the accompanying figures. In the figures, the left-most digit(s) of a reference number identifies the figure in which the reference number first appears. The same numbers are used throughout the drawings to reference like features and components.

### DETAILED DESCRIPTION

Disclosed herein are technologies related to a tool for medical diagnostics based upon material composition of internal organs and tissues of a living creature (such as a human).

Each human is made up of various identifiable organs and tissues. Examples of organs include the heart and lungs. Of course, organs and tissues are composed of various molecules. On average, each identifiable organ and tissue is composed of a specific recipe of ingredients. That is, each body organ/tissue has a determinable composition of material (i.e., material composition). Such material includes elements, molecules, and structures and/or combinations thereof.

Using an implementation of medical diagnostic tool discussed herein, a medical professional (e.g., physician) may make a medical diagnosis of the patient based upon comparing and contrasting the material composition of particular organs (and tissues) over time. For example, a baseline analysis is performed on the heart of a patient and later another analysis of the heart is performed. The material compositions of the two analyses are compared and contrasted. Significant variances are noted. A consultation of a variance-to-diagnosis database indicates a likelihood of a particular diagnosis, such as atherosclerosis.

There is a correlation between the hot spots in the brain and the organs throughout the body. Indication of issues in specific areas of the body can be determined measured and from these indicators the medicines can be taken and within 30 minutes of the time they are taken the body can be retested to show what organs have been affected and if the dosage is too high or too low.

Of course, in order to be a viable medical diagnostic procedure, an example medical diagnostic tool described herein does not destroy or harm the organs and tissues that are being scanned. Most especially, internal organs and tissues. In one or more implementations described herein, the non-destructive and non-harmful means of determining the material composition of internal organs and tissues involve a measurement of the molecular or elemental frequencies found in such organs and tissues. Of course, other implementations may use other techniques for determining material composition non-destructively.

While implementations described herein are focused primarily on living subjects, the technology described herein may be useful with deceased bodies as well. Most especially, the technology will be useful for forensic purposes. Since the techniques described herein are non-destructive and non-invasive, a cause-of-death and/or pathology may be determined without tainting the organs and tissues under study.

### Typical Human Body

The elemental composition of a typical human body can be looked at from the point of view of either mass composition or atomic composition. To illustrate both views, the adult male human body is approximately 57% water, and water is 11% hydrogen by mass but 67% hydrogen by atomic percent. Thus, most of the mass of the human body is oxygen, but most of the atoms in the human body are hydrogen atoms. Being able to define in detail hydrogen in its singular form will provide information on the possible overlapping of genetic codes that may be triggering the malady. Working up through the sequential elemental chart, it can be determined where and what structures have weak transmission values. This is much like an electrical system and finding the paths of highest and lowest resistance using frequency as the impedance within the genetic code sequence the model will no longer be for averaged medical review of the human body but for individual medical applications to treat exactly what area has been affected.

Body composition may also be analyzed in terms of elemental or molecular type (e.g., water, protein, connective tissue, fats (or lipids) apatite (in bones), carbohydrates (such as glycogen and glucose) and DNA. In terms of tissue type, the body may be analyzed into water, fat, muscle, bone, etc.

In the example case of a typical human body discussed below are the materials within a 154-pound person.

TABLE 1

| | | | | Element would | | |
|---|---|---|---|---|---|---|
| | Mass of element | Volume of | | comprise a cube | | |
| | in a 70-kg person | purified | | this long | | |
| Element | 154 pounds | element | | on a side | | OHM's |
| oxygen | 43 kg * 94.7988 lbs. | 37 | L | 33.5 | cm | .02674 |
| carbon | 16 kg * 35.27 lbs. | 7.08 | L | 19.2 | cm | .0005 |
| hydrogen | 7 kg * 15.43 lbs. | 98.6 | L | 46.2 | cm | .001 |
| nitrogen | 1.8 kg * 3.96 lbs. | 2.05 | L | 12.7 | cm | M X 100/6 |
| calcium | 1.0 kg * 2.205 lbs. | 645 | mL | 8.64 | cm | 1.55 |
| phosphorus | 780 g * 27.51 oz. | 429 | mL | 7.54 | cm | 1.0E/−17 10/6 |
| potassium | 140 g * 4.94 oz. | 162 | mL | 5.46 | cm | .139 10/6 |
| sulfur | 140 g * 4.94 oz. | 67.6 | mL | 4.07 | cm | 5.0E/−24 10/6 |
| sodium | 100 g * 3.53 oz. | 103 | mL | 4.69 | cm | .21 10/6 |
| chlorine | 95 g * 3.35 oz. | 63 | mL | 3.98 | cm | .0004 |
| magnesium | 19 g * .067 oz. | 10.9 | mL | 2.22 | cm | .226 10.6 |
| iron | 4.2 g * .148 oz. | 0.53 | mL | 8.1 | mm | .0993 10/6 |
| fluorine | 2.6 g * .092 oz. | 1.72 | mL | 1.20 | cm | .2 |
| zinc | 2.3 g * .081 oz. | 0.32 | mL | 6.9 | mm | .166 10/6 |
| silicon | 1.0 g * .035 oz. | 0.43 | mL | 7.5 | mm | 2.52E/−12 10/6 |
| rubidium | 0.68 g * .024 oz. | 0.44 | mL | 7.6 | mm | .0779 10/6 |
| strontium | 0.32 g * .011 oz. | 0.13 | mL | 5.0 | mm | .0762 10/6 |
| bromine | 0.26 g * .009 oz. | 64.2 | μL | 4.0 | mm | 10/−8 |
| lead | 0.12 g * 004 oz. | 10.6 | μL | 2.2 | mm | .0481 10/6 |
| copper | 72 mg * .0025 oz. | 8.04 | μL | 2.0 | mm | 16.78 |
| aluminum | 60 mg * .0021 oz. | 22 | μL | 2.8 | mm | 26.50 |
| cadmium | 50 mg * .0018 oz. | 5.78 | μL | 1.8 | mm | .138 10/6 |
| cerium | 40 mg * .0014 oz. | 4.85 | μL | 1.7 | mm | .0115 10/6 |
| barium | 22 mg * .0007 oz. | 6.12 | μL | 1.8 | mm | .03 10/6 |
| iodine | 20 mg .0007 oz. | 4.06 | μL | 1.6 | mm | 8.0E/−16 10/6 |
| tin | 20 mg * .0007 oz. | 3.48 | μL | 1.5 | mm | .0917 10/6 |
| titanium | 20 mg * .0007 oz. | 4.41 | μL | 1.6 | mm | .0234 10/6 |
| boron | 18 mg .0006 oz. | 7.69 | μL | 2.0 | mm | 1.0E/−12 10/6 |
| nickel | 15 mg * .00052 oz. | 1.69 | μL | 1.2 | mm | .143 10/6 |
| selenium | 15 mg * .00052 oz. | 3.13 | μL | 1.5 | mm | 1.0E/−12 10/6 |
| chromium | 14 mg * .00049 oz. | 1.95 | μL | 1.3 | mm | .0774 10/6 |
| manganese | 12 mg * .00042 oz. | 1.61 | μL | 1.2 | mm | .226 10/6 |
| arsenic | 7 mg * .00025 oz. | 1.21 | μL | 1.1 | mm | .0345 10/6 |
| lithium | 7 mg * .00025 oz. | 13.1 | μL | 2.4 | mm | 92.8 |
| cesium | 6 mg * .00021 oz. | 3.2 | μL | 1.5 | mm | .0489 10/6 |
| mercury | 6 mg * .00021 oz. | 0.44 | μL | 0.8 | mm | 9.8 × 10−7 |
| germanium | 5 mg * .00018 oz. | 0.94 | μL | 1.0 | mm | 1.45E/−8 10/6 |
| molybdenum | 5mg * .00018 oz. | 0.49 | μL | 0.8 | mm | .187 10/6 |
| cobalt | 3 mg * .00011 oz. | 0.34 | μL | 0.7 | mm | .172 10/6 |
| antimony | 2 mg * .00007 oz. | 0.30 | μL | 0.7 | mm | .0288 10/6 |
| silver | 2 mg * .00007 oz. | 0.19 | μL | 0.6 | mm | 15.87 |
| niobium | 1.5 mg * .000053 oz. | 0.18 | μL | 0.6 | mm | .0693 10/6 |
| zirconium | 1 mg * .0000352 oz. | 0.15 | μL | 0.54 | mm | .0236 10/6 |
| lanthanium | 0.8 mg * .0000281 oz. | 0.13 | μL | 0.51 | mm | .0126 10/6 |
| gallium | 0.7 mg * .0000246 oz. | 0.12 | μL | 0.49 | mm | .0678 10/6 |
| tellurium | 0.7 mg * .0000246 oz. | 0.11 | μL | 0.48 | mm | 2.0E/−6 10/6 |
| yttrium | 0.6 mg * .0000211 oz. | 0.13 | μL | 0.51 | mm | .0166 10/6 |
| bismuth | 0.5 mg * .0000176 oz. | 51 | nL | 0.37 | mm | .00867 10/6 |
| thallium | 0.5 mg * .0000176 oz. | 42 | nL | 0.35 | mm | .0617 10/6 |
| indium | 0.4 mg * .000014 oz. | 55 | nL | 0.38 | mm | .197 10/6 |
| gold | 0.2 mg * .000007 oz. | 10 | nL | 0.22 | mm | 22.14 |
| scandium | 0.2 mg * .000007 oz. | 67 | nL | 0.41 | mm | 1.77 10/6 |
| tantalum | 0.2 mg * .000007 oz. | 12 | nL | 0.23 | mm | 1.3 10/−7 |
| vanadium | 0.11 mg * .0000038 oz. | 18 | nL | 0.26 | mm | .0489 10/6 |
| thorium | 0.1 mg * .0000035 oz. | 8.5 | nL | 0.20 | mm | .0653 10/6 |
| uranium | 0.1 mg * .0000035 oz. | 5.3 | nL | 0.17 | mm | .038 10/6 |
| samarium | 50 μg * .000001764 oz. | 6.7 | nL | 0.19 | mm | 8.800 MO |
| beryllium | 36 μg * .00000127 oz. | 20 | nL | 0.27 | mm | 35.6 |
| tungsten | 20 μg * .00000070548 oz. | 1.0 | nL | 0.10 | mm | .189 10/6 |

Furthermore, each organ/tissue in the human body is comprised of molecules formed from one or more of the elements in Table 1.

While the tissue, bones and organs are made up of unique cellular structures and the entire human body started with two halves of the genetic code from the mother and father, which in turn combined, split and multiplied into the being that we are, there is far more to the transitions than just the point of origin to the person as a whole. It is true that each human comes from the combination of our parents but when you look at the genetic codes in combination, they represent a binary sequence of material passed through the ancestral lineage of each individual.

The cells split, but with each split, there is a taking in of molecular material at the atomic level. The cell takes in energy in the form of hydrogen, oxygen, water, carbon, etc. and these molecules fill in the blanks for the genetic strands. If a genetic strand is magnified, clusters of molecules will be seen that create the connection points along the strands. Taking the strands down to a singular molecular level you will find that the strands are made up of the elements that combined at the outer ring of the molecule. The higher the concentration of molecules (for example a genetic string with a single hydrogen atom bridging the gap between two higher density molecules has a lower potential for transmission of energy) this connection will not have the conductive properties needed to transmit at high level of energy and will severe the connection. This causes a repeated cycle of cells sharing the wrong energy/frequency levels. This is the start of a wild cell forming.

The formation of this miss coded cell, if it grows to a recognizable mass will transmit an energy signal that the body will recognize and even though it is in the wrong place, the body will accept the anomalous mass and feed it. The issue that came from a weak transmission point is now terminal. Remission of a cancerous mass can be achieved in the following manner; one works from the largest mass back toward the cellular point of origin, but we monitor the point of origin and one cellular step below this combination. Determining the exact molecular point of origin and increasing the particular molecular elements needed to reform the genetic strand will stop the cancer growth. This is why the design of the programming and resetting the ranges on current devices is the first step to total individual human body medical oversight.

Determining Material Composition using Matter Oscillation

To have an effective medical diagnostic effect, an example tool described herein presumes that a non-normal organ or tissue will have a different material composition from that of a normal organ or tissue. As used herein, a non-normal organ or tissue is generally abnormal because of disease, illness, stress, chemicals, electrical or other malfunctions in that organ, tissue or other areas of the body.

In addition or in the alternative, elemental composition may be employed to aid in medical diagnosis. That is, the tool may gather data about the elements (and/or the molecules) that comprise a human's organ or tissue as part of a medical diagnosis.

Matter vibrations involve different kinds of motion for molecules that include translational motion (i.e., when the whole molecule goes in the same direction) and rotational motion (i.e., when the molecule spins like a top). As used herein, matter oscillation includes both translational vibration and rotation to create unique frequency outputs synonymous with the three dimensional size of each identifiable form of matter (e.g., element, molecule, etc.).

For example, a vibrational motion for a molecule is when the bonds between atoms within a molecular move. Think of the atoms as round balls that are attached by a spring that can stretch back and forth. An example of this motion is “stretching”, the simplest example of a vibration for a molecule and occurs between just two atoms.

There is a relationship between the energy of a matter oscillation and its material composition. Indeed, the relationship is one of identity. That is, the composition of material (e.g., molecules) may be determined from its measured oscillation. Frequency is a measure of the matter oscillation.

All matter has a potential energy and this energy can be determined by the frequency being transmitted. Each molecule has a frequency that correlates with the three dimensional size of the molecule. As the potential energy increases so does the molecular size and in turn the frequency will increase in size. In the case of for example of one hydrogen molecule is 0.1 nanometer Diameter so would indicate that the frequency for hydrogen is 0.1 nanometers.

Using the same equations in nanometers we can convert the values of the nanometers into hexadecimal configurations in a computer program and run exact sequential values for molecules and the frequencies that each molecule is part of and determine the individual points of weakness in any cell, for that matter any type molecular structure, for example, can be tested to determine the rate of decay and life cycle expected (not just the human body but buildings, airplanes, bridges, ships, oil rigs and pipelines any type structural support systems.) Molecular interfaces of any kind due to the composition of the elements used are in the human body and every form of matter surrounding us.

With regard to the relationship of material composition and its oscillation, consider the following: The lowest vibrational transitions of diatomic molecules approximate the quantum harmonic oscillator and can be used to imply the bond force constants for small oscillations. The following is a sampling of transition frequencies from the n=0 to n=1 vibrational level for diatomic molecules and the calculated force constants.

| | | Force constant |
|---|---|---|
| Molecule | Frequency ×10¹³ Hz | N/m |
| HF | 12.4* | 970 |
| HCl | 8.66 | 480 |
| HBr | 7.68 | 410 |
| HI | 6.69 | 320 |
| CO | 6.42 | 1860 |
| NO | 5.63 | 1530 |

* From vibrational transition 4138.52 cm⁻¹ in Herzberg's

### Operation of an Example Tool for Medical Diagnostics

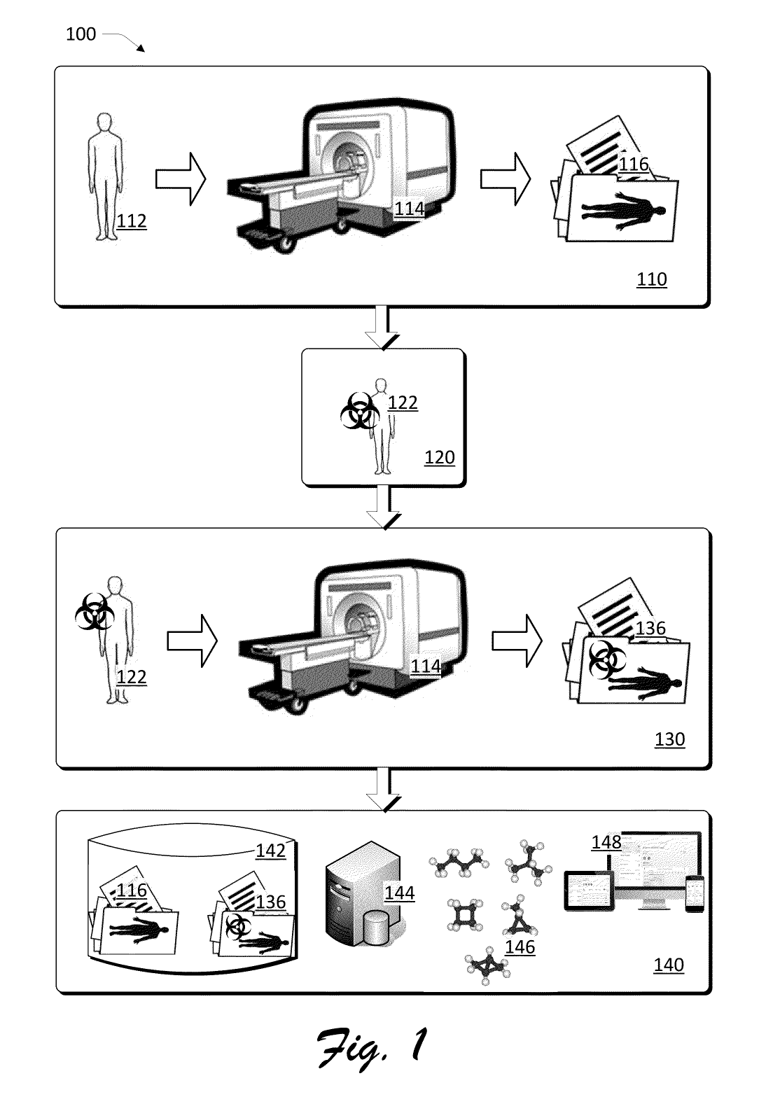

FIG. 1 illustrates an example operation set 100 for implementing, at least in part, the technology described herein. In particular, operation set 100 depicts a medical diagnosis performed by an example medical diagnosis tool as described herein.

Collectively, the example medical diagnostic tool as described herein performs the operation set 100. The example medical diagnostic tool includes, for example, one or more components such a magnetic resonance imaging (MRI) machine 112, a database server 142, a computing system 144, and a diagnosis-report output system 148. In other implementations, the example medical diagnostic tool includes, in part or in whole, by an integrated computing system or a distributed computing system (such as over a network).

At 110, a medical professional establishes a baseline model of a subject's body. More particularly, the baseline model records three-dimensional data of one or more of the subject's organs and tissues. This may be accomplished, for example, by determining the material composition of such organs/tissues when the subject is in a “normal” state. A normal state can also be called healthy or balanced state.

As depicted, operation 110 shows the MRI machine 114 scanning a nominally normal person 112 (i.e., a “healthy” person). From the scan, the organs/tissues of the normal person 112 are identified. Organ/tissue identification is accomplished using conventional or new techniques. The scan of the normal person 112 results in a three-dimensional representation (i.e., map) of the matter oscillation (e.g., vibration and/or rotation) of one or more of the identifiable organs/tissues of the normal person 112. Using an oscillation-to-matter correlation table, the example medical diagnostic tool determines the material composition of each identifiable organs/tissues of the normal person 112. This is stored in a baseline model 116, which is associated with the patient, who is the normal person 112 depicted here.

The baseline model 116 may be created from a single scan of one or more organs/tissues or from statistical analysis (e.g., mean or median) over several scans. Of course, the scans that establish the baseline model 116 presumably occur when the nominally normal person 112 is actually healthy, drug-free, disease-free, and under minimal stress.

For example, a breakdown of the baseline model 116 of the heart of the normal person 112 (presuming 154 lbs. of body weight) may include the following material composition:

For example, the typical human heart is made up of the following elements, in the correct combination the heart will operate at specific energy levels and produce sine waves in peak-to-peak fashion, which is the overall combination of the elements listed below. If these individual elements become unbalanced, the heart will form an imbalance in the erythematic functions and using the process mentioned in this document the elemental imbalance can be found.

Elements

| | |
|---|---|
| 1. Oxygen | (65%) |
| 2. Carbon | (18%) |
| 3. Hydrogen | (10%) |
| 4. Nitrogen | (3%) |
| 5. Calcium | (1.5%) |
| 6. Phosphorus | (1.0%) |
| 7. Potassium | (0.35%) |
| 8. Sulfur | (0.25%) |
| 9. Sodium | (0.15%) |
| 10. Magnesium | (0.05%) |
| 11. Copper, Zinc, Selenium, Molybdenum, Fluorine, | (0.70%) |
| Chlorine, Iodine, Manganese, Cobalt, Iron | |
| 12. Lithium, Strontium, Aluminum, Silicon, Lead, | (trace amounts). |
| Vanadium, Arsenic, Bromine | |

The following reference provides some guidance on the material composition of the typical human heart: H. A. Harper, V. W. Rodwell, P. A. Mayes, Review of Physiological Chemistry, 16th ed., Lange Medical Publications, Los Altos, Calif. 1977.

At 120, some event or events occur that converts the otherwise normal person into a nominally abnormal person 122 (i.e., unhealthy person). Such events may include stress inductions, exposure to chemical, biological, or radiological influences, discovery of a medical condition (such as cancer), or anything else that will cause a biological or medical difference between the normal person 112 and the abnormal person 122.

At 130, a medical professional generates a new or subsequent model of a subject's body, which presumably occurs after the event(s) of 120. More particularly, this subsequent model includes three-dimensional data of one or more of the subject's organs and tissues. This may be accomplished, for example, by determining the material composition of such organs/tissues when the subject is in its “abnormal” state. The abnormal state can also be called unhealthy or unbalanced state.

As depicted, operation 130 shows the MRI machine 114 scanning a nominally abnormal person 122 (i.e., an “unhealthy” person). From the scan, the organs/tissues of the abnormal person 122 are identified. The scan of the abnormal person 122 results in a three-dimensional representation (i.e., map) of the matter oscillation (e.g., vibration and/or rotation) of one or more of the identifiable organs/tissues of the normal person 122. Using an oscillation-to-matter correlation table, the example medical diagnostic tool determines the material composition of each identifiable organs/tissues of the abnormal person 122. This is stored in a subsequent model 136, which is associated with the patient, who is both the normal person 112 and the abnormal person 122.

The subsequent model 136 may be created from a single scan of one or more organs/tissues or from statistical analysis (e.g., mean or median) over several scans. Of course, the scans that produce the subsequent model 136 presumably occur when the nominally abnormal person 122 is actually unhealthy, drug-influence, diseased, under abnormal stress, or the like.

For example, a breakdown of the subsequent model 136 of the heart of the abnormal person 122 (presuming 154 lbs. of body weight) may include the following material composition:

Elements

| | |
|---|---|
| 1. Oxygen | (65%) |
| 2. Carbon | (25%) |
| 3. Hydrogen | (10%) |
| 4. Nitrogen | (3%) |
| 5. Calcium | (2.5%) |
| 6. Phosphorus | (1.5%) |
| 7. Potassium | (0.35%) |
| 8. Sulfur | (0.25%) |
| 9. Sodium | (0.30%) |
| 10. Magnesium | (0.05%) |
| 11. Copper, Zinc, Selenium, Molybdenum, Fluorine, | (0.70%) |
| Chlorine, Iodine, Manganese, Cobalt, Iron | |
| 12. Lithium, Strontium, Aluminum, Silicon, Lead, | (trace amounts) |
| Vanadium, Arsenic, Bromine | |

The above would potentially become an enlarged heart. Doing baseline monitoring and comparing the abnormal heart to the baseline normal heart and then inputting the medicines being taken (these could be depleting the absorption capabilities of the heart, and other major organs for that matter) we can instantaneously have a reference point and actual information to correct the issue. In correcting the issue, the process will allow the doctor or technician a real-time overview and will offer a way to see how medications are interacting in real time, in the initial sequence of development

At 140, the operation includes the database server 142 with a database containing the baseline model 116 with the subsequent model 136, the computing system 144 records of the differences in the material composition (between the baseline and subsequent models), and the diagnosis-report output system 148. The diagnosis-report output system 148 includes one or more distributed presentation devices (such as a computer screen, tablet screen, or a smartphone screen) with an associated user interface to provide the medical data resulting from the example medical diagnosis tool described herein.

As part of the analysis of operation 140, the baseline model 116 with the subsequent model 136 of the patient are compared and/or contrasted. This analysis may cover the entire body or select areas of the body. This analysis may be focused on particular organs/tissues or portions thereof. For illustration purposes only, the discussion hereinafter focuses on one organ for the analysis. Of course, a person of ordinary skill in the art understands that such analysis may be limited to parts of an organ or tissue or cover several organs or tissues.

From the comparing and/or contrasting, differences in the material composition of a subject organ is determined. Molecules 146 in FIG. 1 represent that difference.

The computing system 144 notes these differences to the medical professional. In some implementations, the data regarding the raw differences in material composition in the subject organ is reported via the diagnosis-report output system 148 to the medical professional and/or the patient. In some implementations, the data regarding the raw differences in matter oscillation (or energy/Frequency) in the subject organ is reported via the diagnosis-report output system 148 to the medical professional and/or the patient.

In some implementations, the medical professional exercises their medical judgment. Based upon the differences in material composition, the medical professional may diagnosis a medical condition and perhaps suggests a treatment. In these implementations, the medical diagnosis (and/or suggested treatments) is reported or documented via the diagnosis-report output system 148 to the medical professional and/or the patient.

In other implementations, the computing system 144 may automatically generate a report regarding the likelihood of particular medical diagnoses. This may be accomplished (in whole or in part) by heuristics provided by medical professionals. In addition or in the alternative, this may be accomplished by using machine-learning techniques. In these implementations, the medical diagnosis (and/or suggested treatments) is reported or documented via the diagnosis-report output system 148 to the medical professional and/or the patient.

In still other implementations, the computing system 144 may suggest medical diagnosis (and treatments) to the medical professional (via the output system 148). From this, the medical professional may use these suggestions to make their own medical judgment with regard to the appropriate diagnosis and treatment thereof.

With the various implementations discussed above, the example medical diagnostic tool (1) provides the diagnostic data upon which a human (e.g., the medical professional) bases their final medical diagnosis, (2) determines an actual medical diagnosis based upon the diagnostic data that it generated or acquired; or (3) shares the diagnostic duties with the human (e.g., the medical professional).

In still other implementations, other medically relevant data may be considered in making the diagnosis and treatment determinations. Such data may include, for example, vital signs (e.g., body temperature, pulse rate, blood pressure, and respiratory rate); known medical conditions; medications; allergies; blood test results; patient self-report of condition; medical history; electrocardiogram (ekg); electroencephalogram (eeg); X-ray; diet; exercise; etc.

### Example System

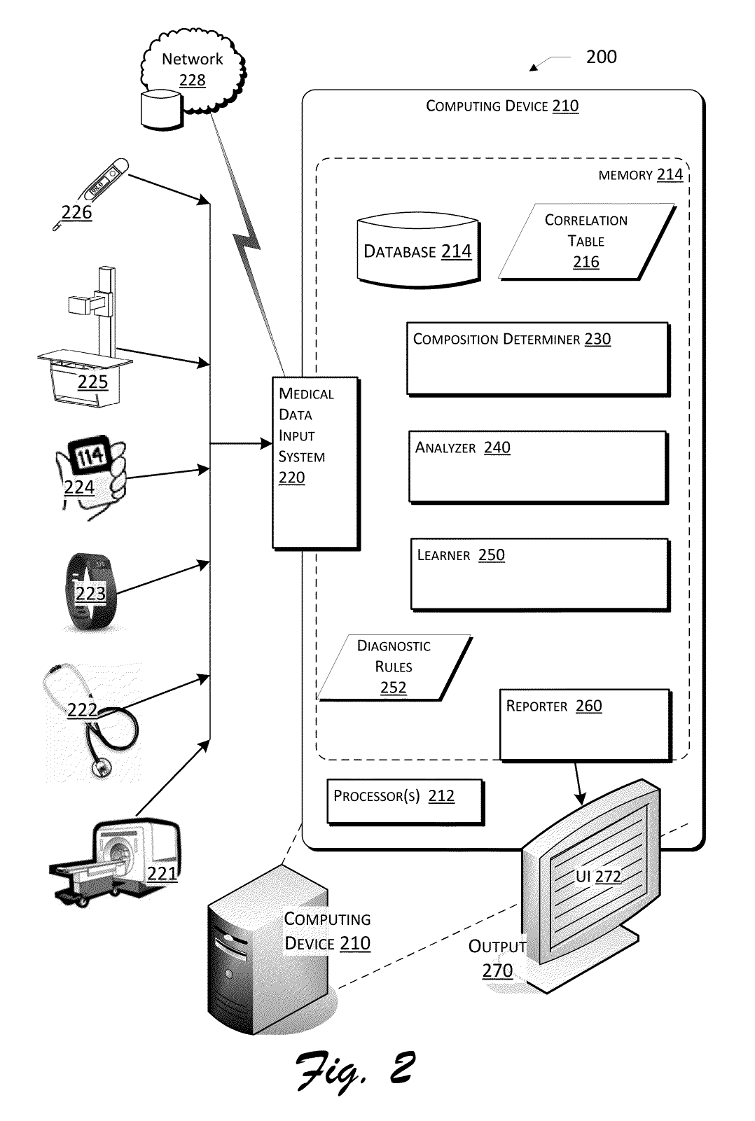

FIG. 2 illustrates example system 200 for implementing the technology described herein. The system 200 includes one or more computing devices 210, a medical data input system 220, and output system 270.

The example system includes one or more computing devices 210, various medical data sources 221-226 and a network 228. Alternatively, one may implement the components of the example system 100, at least in part, in more integrated systems.

As shown, the computing device 210 includes one or more processor(s) 212, a memory 214, a medical-data input system 220, a composition determiner 230, an analyzer 240, a learner 250, and a reporter 260. These functional components may be separate or some combination of hardware units. Alternatively, the components may be implemented, at least in part, via software and thus be stored in the memory 214 and executed by the processors 212.

Using the system 200, the medical-data input system 220 acquires information about a subject using one or more various medical data sources 221-226. As depicted, the sources of medical data includes an MRI machine 221, vital signs monitors (such as stethoscope) 222, quantified-self devices (such as FITBIT™ monitors) 223, blood glucose monitor 224, X-ray machine 225, and thermometer 226. Of course, medical data can be derived from other medical equipment and/or via the network 228 (or so-called cloud).

All medical equipment has a reference base that deals in some way with the elemental makeup of the body, but we are currently just using the empirical data and not taking into consideration the over production of glucose for instance. Defining the safe levels is what medical practitioner's do by determining the base of “normal” readings within a curve. The process of determining the elemental values to the most simplistic values and the overall values within the body will help determine not only the increased rates but also what element is being over produced, where in the body and what drivers are taking place to allow these changes. This can be determined to the genetic level (as indicated earlier in the document) and the energy flows can be boosted within the gene strands to strengthen the flow of energy transmission or can be weakened to slow the genetic code transmission in the over production of the glucose.

The medical data used herein includes, at the very least, data regarding the matter oscillation of one or more organs/tissues of the subject. In some implementations, other medical data from one or more other sources may be used and correlated with the matter oscillation data.

Initially, medical data from a nominally normal person is gathered. From this data, the organs/tissues of the normal person are identified. This data, a three-dimensional representation (i.e., map) of the matter oscillation (e.g., vibration/frequency and/or rotation) of one or more of the identifiable organs/tissues of the normal person is produced. Using an oscillation-to-matter correlation table 216, the composition determiner 230 of the system 200 determines the material composition of each identifiable organs/tissues of the normal person. This is stored in a baseline model in a database 214. The baseline model is associated with the patient, who is the normal person discussed herein.

Presumably, some event or events occur that converts the otherwise normal person into a nominally abnormal person (i.e., unhealthy person). Such events may include stress inductions, food intake or exposure to chemical, biological, or radiological influences, discovery of a medical condition (such as cancer), or anything else that will cause a biological or medical difference between the normal person and the abnormal person.

Next, medical data from the nominally abnormal person is gathered. From this data, the organs/tissues of the abnormal person are identified. This data, a three-dimensional representation (i.e., map) of the matter oscillation (and perhaps other medical data) of one or more of the identifiable organs/tissues of the abnormal person is produced. Using the oscillation-to-matter correlation table 216, the composition determiner 230 of the system 200 determines the material composition of each identifiable organs/tissues of the abnormal person. This is stored in a subsequent model in the database 214. The subsequent model is associated with the patient, who is both the normal and abnormal person discussed herein. The three dimensional overview is of the individual elemental structures showing the specific masses of cells that form the transmission and response phases to allow any organ to correctly operate. This application will determine what elements have been depleted or over absorbed and allow for exact corrective measures.

The analyzer 240 analyzes the baseline model and the subsequent model (which are found in the database 214 or perhaps in the network 228). The analyzer 240 records (or highlights or notes) the differences in the material composition between the baseline and subsequent models.

The analyzer 240 compares and/or contrasts the baseline and subsequent models. This analysis may cover the entire body or select areas of the body. This analysis may be focused on particular organs/tissues or portions thereof. For illustration purposes only, the discussion hereinafter focuses on one organ for the analysis. Of course, a person of ordinary skill in the art understands that such analysis may be limited to parts of an organ or tissue or cover several organs or tissues. From the comparing and/or contrasting, differences in the material composition of a subject organ is determined.

The reporter 260 notes these differences to the medical professional. In some implementations, the data regarding the raw differences in material composition in the subject organ is reported via a user interface (UI) 272 of the output system 270 to the medical professional and/or the patient. In some implementations, the data regarding the raw differences in matter oscillation (or energy) in the subject organ is reported via the user interface (UI) 272 of the output system 270 to the medical professional and/or the patient.

In some implementations, the medical professional exercises his/her medical judgment. Based upon the differences in material composition, the medical professional may diagnosis a medical condition and perhaps suggests a treatment. In these implementations, the medical diagnosis (and/or suggested treatments) is reported or documented via the user interface (UI) 272 of the output system 270 to the medical professional and/or the patient.

In other implementations, the reporter 260 may automatically generate a report regarding the likelihood of particular medical diagnoses. This may be accomplished (in whole or in part) by heuristics provided by medical professionals. In addition or in the alternative, this may be accomplished by using machine-learning techniques. In these implementations, the medical diagnosis (and/or suggested treatments) is reported or documented via the user interface (UI) 272 of the output system 270 to the medical professional and/or the patient.

In still other implementations, the reporter 260 may suggest medical diagnosis (and treatments) to the medical professional (via the output system 270). From this, the medical professional may use these suggestions to make his/her own medical judgment with regard to the appropriate diagnosis and treatment thereof.

A diagnostic rules dataset 252 stores the human-supplied heuristics and/or machine-learned rules that aids in a machine-aided or automatic diagnosis. The diagnostic rules dataset 252 include correlations involving material composition (and/or matter oscillation) and diagnosis steps and/or possible treatments. This dataset 252 may include correlations involving other medical data of the subject, like those derived from various sources 221-226.

The output system 270 includes one or more presentation devices (such as a computer screen, tablet screen, or a smartphone screen) with an associated user interface 272 to provide the medical data resulting from the example medical diagnosis tool described herein.

Stated in another way, the system 200 may be described as including (for example) the following:
an input system (e.g., system 220) configured to obtain medical data about a particular internal organ or tissue of a living subject;
a composition determiner (e.g., composition determiner 230) configured to determine, based upon the obtained medical data, composition of material composition of the particular internal organ or tissue of the subject;
an analyzer (e.g., analyzer 240) configured to determine differences in material composition of the particular internal organ or tissue of the living subject by comparing and/or contrasting the particular internal organ or tissue at one point in time to the same particular internal organ or tissue at another point in time;
a reporter (e.g., reporter 260 and output system 270) configured to report the determined differences.

### Operations for Medical Diagnostic Tool

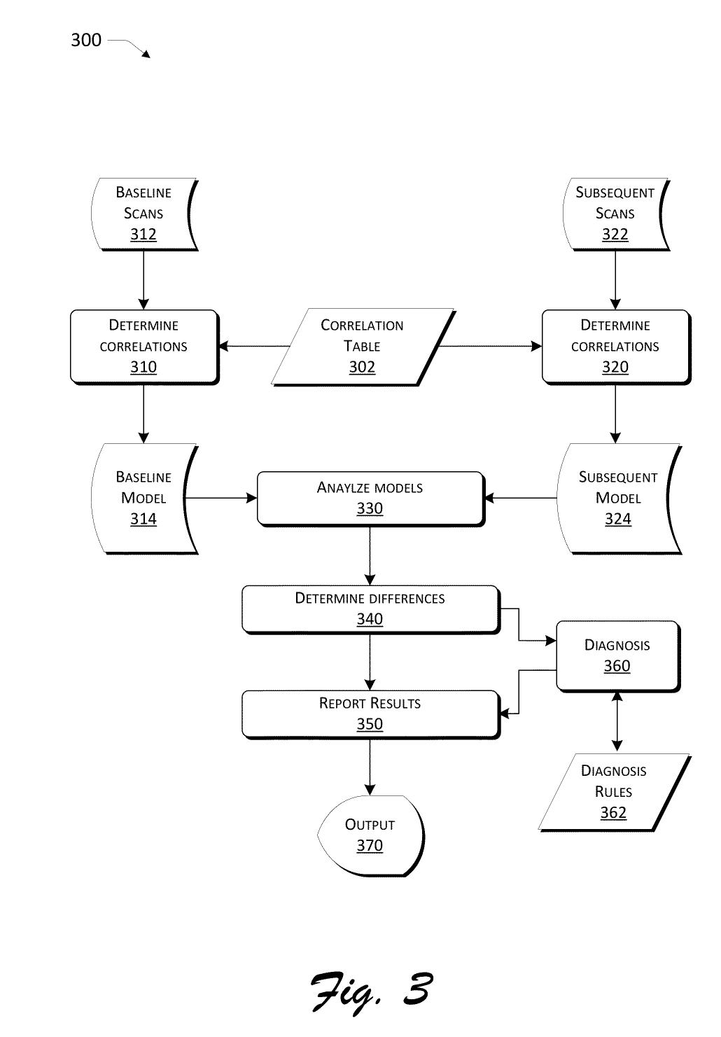

FIG. 3 illustrates an example process 300 for implementing, at least in part, the technology described herein. In particular, process 300 depicts the operations of the medical diagnostic tools described herein. The process 300 is performed by, for example, the system 200.

At 310, the system obtains medical data (e.g., via baseline scans 312) from a nominally normal person. From this data, the organs/tissues of the normal person are identified. Using an oscillation-to-matter correlation table 302, the system determines the material composition of each identifiable organs/tissues of the normal person. Also, a three-dimensional representation (i.e., map) of the matter oscillation (e.g., vibration and/or rotation) of one or more of the identifiable organs/tissues of the normal person is produced. This is a baseline model 314 of the subject. The baseline model 314 is associated with the patient, who is the normal person discussed herein.

Presumably, some event or events occur that converts the otherwise normal person into a nominally abnormal person (i.e., unhealthy person).

At 320, the system obtains medical data (e.g., via subsequent scans 322) from the nominally abnormal person. From this data, the organs/tissues of the abnormal person are identified. Using the oscillation-to-matter correlation table 302, the system determines the material composition of each identifiable organs/tissues of the abnormal person. Also, a three-dimensional representation (i.e., map) of the matter oscillation (e.g., vibration, oscillation/frequency and/or rotation) of one or more of the identifiable organs/tissues of the abnormal person is produced. This is a subsequent model 324 of the subject. The subsequent model 324 is associated with the patient, who is both the normal person and abnormal person discussed herein.

Collectively, operations 310 and 320 may be described, for example, in this manner: obtaining medical data about a particular internal organ or tissue of a living subject; determining material composition of the particular internal organ or tissue of a living subject, the determining being based upon the obtained medical data; and generating a baseline model of the particular internal organ or tissue at the one point in time and a subsequent model of the particular internal organ or tissue at the later point in time, wherein each model is a three-dimensional data representation of the material composition of the particular internal organ or tissue at their respective time.

At 330, the system analyzes the baseline model 314 and the subsequent model 324. The system compares and/or contrasts the baseline and subsequent models. This analysis may cover the entire body or select areas of the body. This analysis may be focused on particular organs/tissues or portions thereof. For illustration purposes only, the discussion hereinafter focuses on one organ for the analysis. Of course, a person of ordinary skill in the art understands that such analysis may be limited to parts of an organ or tissue or cover several organs or tissues.

At 340, the system determines the differences in the material composition of a subject organ based upon the comparing and/or contrasting of operation 330.

Collectively, operations 330 and 340 may be described, for example, in this manner: finding differences in material composition of the particular internal organ or tissue of the living subject by comparing and/or contrasting the baseline and subsequent models of the particular internal organ or tissue.

At 350, the system records (or highlights or notes) the differences in the material composition between the baseline and subsequent models. The system notes these differences to the medical professional. In some implementations, the data regarding the raw differences in material composition in the subject organ is reported via output 370 to the medical professional and/or the patient. In some implementations, the data regarding the raw differences in matter oscillation (or energy) in the subject organ is reported via output 370 to the medical professional and/or the patient.

In some implementations, the medical professional exercises her medical judgment. Based upon the differences in material composition, the medical professional may diagnosis a medical condition and perhaps suggests a treatment. In these implementations, the medical diagnosis (and/or suggested treatments) is reported or documented via output 370 to the medical professional and/or the patient.

At 360, in some implementations, the system may automatically generate a report regarding the likelihood of particular medical diagnoses. This may be accomplished (in whole or in part) by heuristics provided by medical professionals. In addition or in the alternative, this may be accomplished by using machine-learning techniques. In these implementations, the medical diagnosis (and/or suggested treatments) is reported or documented via output 370 to the medical professional and/or the patient.

At 360, in still other implementations, the system may suggest medical diagnosis (and treatments) to the medical professional (via the output system 270). From this, the medical professional may use these suggestions to make her own medical judgment with regard to the appropriate diagnosis and treatment thereof.

A diagnostic rules dataset 362 stores the human-supplied heuristics and/or machine-learned rules that aids in a machine-aided or automatic diagnosis. The diagnostic rules dataset 362 include correlations involving material composition (and/or matter oscillation) and diagnosis steps and/or possible treatments. This dataset 362 may include correlations involving other medical data of the subject, like those derived from various sources.

Collectively, operations 350 and 360 may be described, for example, in this manner: facilitating a medical diagnosis of the subject, the medical diagnosis being based, at least in part, upon a correlation between the differences in material composition of the particular internal organ or tissue and medical diagnostic data and reporting the medical diagnosis of the subject.

### Exemplary System

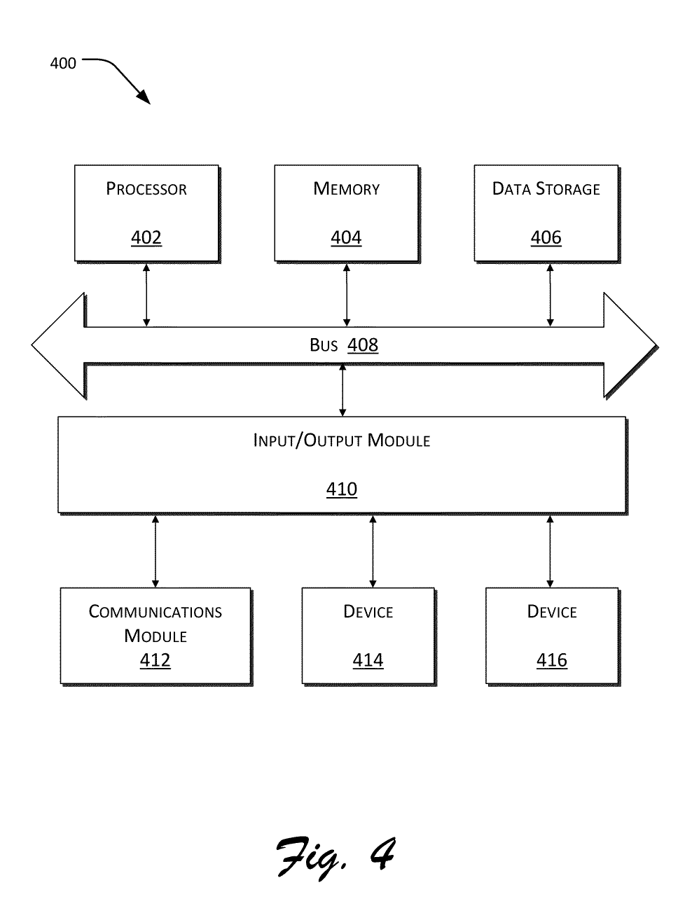

FIG. 4 is a high-level block diagram illustrating an example computer system 400 suitable for implementing the technologies described herein. In certain aspects, the computer system 400 may be implemented using hardware or a combination of software and hardware.

The illustrated computer system 400 includes a processor 402, a memory 404, and data storage 406 coupled to a bus 408 or other communication mechanism for communicating information. An input/output (I/O) module 410 is also coupled to the bus 408. A communications module 412, a device 414, and a device 416 are coupled to the I/O module 410.

The processor 402 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information. The processor 402 may be used for processing information. The processor 402 can be supplemented by, or incorporated in, special purpose logic circuitry.

The memory 404 may be Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device used for storing information, a computer program, and/or instructions to be executed by the processor 402. They memory 404 may store code that creates an execution environment for one or more computer programs used to implement technology described herein.

A computer program as discussed herein does not necessarily correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Unless indicated otherwise by the context, a module refers to a component that is hardware, firmware, and/or a combination thereof with software (e.g., a computer program.) A computer program as discussed herein does not necessarily correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

The instructions may be implemented in one or more computer program products, i.e., one or more modules of computer program instructions encoded on one or more computer readable media for execution by, or to control the operation of, the computer system 400, and according to any method well known to those of skill in the art. The term “computer-readable media” includes computer-storage media. For example, computer-storage media may include, but are not limited to, magnetic storage devices (e.g., hard disk, floppy disk, and magnetic strips), optical disks (e.g., compact disk (CD) and digital versatile disk (DVD)), smart cards, flash memory devices (e.g., thumb drive, stick, key drive, and SD cards), and volatile and non-volatile memory (e.g., random access memory (RAM), read-only memory (ROM)).

The data storage 406 may be a magnetic disk or optical disk, for example. The data storage 506 may function to store information and instructions to be used by the processor 402 and other components in the computer system 400.

The bus 408 may be any suitable mechanism that allows information to be exchanged between components coupled to the bus 508. For example, the bus 408 may be transmission media such as coaxial cables, copper wire, and fiber optics, optical signals, and the like.

The I/O module 410 can be any input/output module. Example input/output modules 410 include data ports such as Universal Serial Bus (USB) ports.

The communications module 412 may include networking interface cards, such as Ethernet cards and modems.

The device 414 may be an input device. Example devices 414 include a keyboard, a pointing device, a mouse, or a trackball, by which a user can provide input to the computer system 400.

The device 416 may be an output device. Example devices 416 include displays such as cathode ray tubes (CRT) or liquid crystal display (LCD) monitors that display information, such as web pages, for example, to the user.

### Additional and Alternative Implementation Notes

The original test baseline will be done with a known healthy individual, a known unhealthy individual and the figures of the two would become the upper and lower control ranges. From this point each individual would have their own scans and determine what organs are being affected, what the medicines are doing to other organs and how to treat this individual to for the exact issues falling outside the base line. The overall positive is that every time you go to the doctor that run your previous tests against the ones completed at this visit and can tell you how well your medicines are working, what is too high or too low and see the onset of potential diseases and treat the individual with the exact medicines needed and see an immediate effect of the medicines by having the patient take the medicine wait an hour and then run a new test no more guessing about what works and what side effects that currently have weeks or months to do damage using the practice that is now accepted.

Unlike conventional approaches, the technologies described herein allow for an analysis of the composition of the internal organs and tissues of a living creature (e.g., a human). Unlike a traditional medical imaging (such as is typically done with MRIs), the technologies described herein determine actual composition of the organs and tissue of a living organism.

The human body, for example, reacts to stimuli and electrochemical baseline frequency pulses that create programmed responses within the body. Either these responses reflect balanced or imbalanced interactions transmitted and received within control perimeters of what is expected to be seen by these actions.

When the body is operating within the expected control range there is a balanced response (e.g., normal) that can be tracked and used to measure against the times that the body is in a state of discomfort (e.g., abnormal). Cells like any other molecular structure react as groups of critical mass compositions forming the unique required cell structures to become any given organ or structure in the body. These groups each have indicators that determine the matter vibration and these frequency variations activate the strands of information at that interaction point at the chromosome/molecular level. These inputs, outputs and frequency levels are programmed into our system and the technology described herein uses these to monitor and control our functions and rebuilding of our cellular structure.

The inventor has found that all molecular structures operate in specific naturally occurring logarithms. This is true of the human cellular structures too. Human bodies produce pressure, temperature, positive and negative energy pulses and unique signature is that the human body recognizes as baseline control levels.

The process for determining the placement and type cell is driven by what was once thought to be transient energy electrical impulses. It is far more complex than that but when the calculations are done using the known frequencies that deliver the exact size molecular energy pulse the result is a unique pattern that programs the cells in accordance to the distance and critical mass connectivity throughout the body. For example, the interaction and program for a fingernail is at a vibrational rate determined by, Distance, Temperature, energy rate (plus or minus 0.0000002), Frequency, Vibration and Time Differentials.

Minute shifts in Frequency will determine the amount of critical mass any cellular structure can support. Within the range of plus or minus 0.000.000.2 can support exact numeric values before either increasing or decreasing in the ability to travel within that specific Frequency and at that point the cells will transition into the next set of cellular mass. In these calculations, Pi sequential values are used and those values have the potential to shift between plus or minus 0.000.000.2 and as the variations shift from the sequence within the range of Frequency to slower or faster energy pulses the cells or molecules will become a changed hybrid of the original while still staying within the natural mathematical constants.

This leads to the irregular or wild cell application. Should the frequency indicator for any area shift and become a critical mass of cells the system will recognize the input shift and allow the change. This allowance creates a “program change” and the body (any molecular structure for that matter) becomes an abnormal normality. This action can be predicted and the imbalance can be reprogrammed at the molecular level to re-align the frequency to energy correlation.

In the above description of exemplary implementations, for purposes of explanation, specific numbers, materials configurations, and other details are set forth in order to better explain the present invention, as claimed. However, it will be apparent to one skilled in the art that the claimed invention may be practiced using different details than the exemplary ones described herein. In other instances, well-known features are omitted or simplified to clarify the description of the exemplary implementations.

The inventor intends the described exemplary implementations to be primarily examples. The inventor does not intend these exemplary implementations to limit the scope of the appended claims. Rather, the inventor has contemplated that the claimed invention might also be embodied and implemented in other ways, in conjunction with other present or future technologies.

Moreover, the word “exemplary” is used herein to mean serving as an example, instance, or illustration. Any aspect or design described herein as exemplary is not necessarily to be construed as preferred or advantageous over other aspects or designs. Rather, use of the word “exemplary” is intended to present concepts and techniques in a concrete fashion. The term “technology,” for instance, may refer to one or more devices, apparatuses, systems, methods, articles of manufacture, and/or computer-readable instructions as indicated by the context described herein.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

These processes are illustrated as a collection of blocks in a logical flow graph, which represents a sequence of operations that can be implemented in mechanics alone or a combination with hardware, software, and/or firmware. In the context of software/firmware, the execution of the instructions on the medium may cause performance of the operations described herein. For example, or more computer-readable media with processor-executable instructions stored thereon which when executed by one or more processors may cause performance of operations described herein.

Note that the order in which the processes are described is not intended to be construed as a limitation, and any number of the described process blocks can be combined in any order to implement the processes or an alternate process. Additionally, individual blocks may be deleted from the processes without departing from the spirit and scope of the subject matter described herein.

The term “computer-readable media” is computer-storage media. For example, computer-storage media may include, but are not limited to, magnetic storage devices (e.g., hard disk, floppy disk, and magnetic strips), optical disks (e.g., compact disk [CD] and digital versatile disk [DVD]), smart cards, flash memory devices (e.g., thumb drive, stick, key drive, and SD cards), and volatile and nonvolatile memory (e.g., RAM and ROM).

What is claimed is:

1. A medical diagnostic tool that facilitates a medical diagnosis, the tool comprising:
an input system configured to obtain medical data about a particular internal organ or tissue of a living subject;
a composition determiner configured to determine, based upon the obtained medical data, composition of material of the particular internal organ or tissue of the subject;
an analyzer configured to determine differences in material composition of the particular internal organ or tissue of the living subject by comparing and/or contrasting the particular internal organ or tissue at one point in time to the same particular internal organ or tissue at another point in time;
a reporter configured to report the determined differences.

2. A medical diagnostic tool according to claim 1, wherein:
the composition determiner is further configured to generate a baseline model of the particular internal organ or tissue at the one point in time and a subsequent model of the particular internal organ or tissue at the later point in time, wherein each model is a three-dimensional data representation of the material composition of the particular internal organ or tissue at their respective time.

3. A medical diagnostic tool according to claim 1, wherein the medical data is derived, at least in part, from one or more scans of the particular internal organ or tissue performed by a magnetic imaging resonance (MRI) machine.

4. A medical diagnostic tool according to claim 1, wherein:
the medical data includes information about matter oscillations within the particular organs or tissue of the living subject;
the composition determiner is further configured to determine the material composition of the particular internal organ or tissue based upon one or more correlations between the matter oscillations and material composition.

5. A medical diagnostic tool according to claim 1, wherein:
the medical data includes information about matter oscillations within the particular organs or tissue of the living subject, wherein the matter oscillations within the particular internal organ or tissue is derived from one or more scans of the particular internal organ or tissue performed by a magnetic imaging resonance (MRI) machine;
the composition determiner is further configured to determine the material composition of the particular internal organ or tissue based upon one or more correlations between the matter oscillations and material composition.

6. A medical diagnostic tool according to claim 1, wherein the composition determiner includes a magnetic resonance imaging (MRI) machine.

7. A medical diagnostic tool according to claim 1, wherein the reporter is further configured to facilitate with a medical diagnosis of the subject, the medical diagnosis being based, at least in part, upon a correlation between the differences in the material composition of the particular internal organ or tissue and medical diagnostic data.

8. A medical diagnostic tool according to claim 1, wherein the reporter is further configured to produce output that is consumable by a human.

9. A medical diagnostic tool according to claim 1, wherein the material composition is selected from a group consisting of elements, molecules, and a combination thereof.

10. One or more computer-readable media storing processor-executable instructions that when executed cause one or more processors to perform operations comprising:
obtaining medical data about a particular internal organ or tissue of a living subject;
determining material composition of the particular internal organ or tissue of a living subject, the determining being based upon the obtained medical data;
finding differences in material composition of the particular internal organ or tissue of the living subject by comparing and/or contrasting the particular internal organ or tissue at one point in time to the same particular internal organ or tissue at another point in time;
facilitating a medical diagnosis of the subject, the medical diagnosis being based, at least in part, upon a correlation between the differences in material composition of the particular internal organ or tissue and medical diagnostic data;
reporting the medical diagnosis of the subject.

11. One or more computer-readable media according to claim 10, wherein the operations further comprise:
generating a baseline model of the particular internal organ or tissue at the one point in time and a subsequent model of the particular internal organ or tissue at the later point in time, wherein each model is a three-dimensional data representation of the material composition of the particular internal organ or tissue at their respective time.

12. One or more computer-readable media according to claim 11, wherein the finding includes comparing and/or contrasting the baseline and subsequent models.

13. One or more computer-readable media according to claim 10, wherein the determining operation is based upon scans of the particular internal organ or tissue performed by a magnetic imaging resonance (MRI) machine.

14. One or more computer-readable media according to claim 10, wherein:
the medical data includes information about matter oscillations within the particular organs or tissue of the living subject;
the determining operation being based upon one or more correlations between the matter oscillations and material composition.

15. One or more computer-readable media according to claim 10, wherein:
the medical data includes information about matter oscillations within the particular organs or tissue of the living subject, wherein the matter oscillations within the particular internal organ or tissue is derived from one or more scans of the particular internal organ or tissue performed by a magnetic imaging resonance (MRI) machine;
the determining operation being based upon one or more correlations between the matter oscillations and material composition.

16. One or more computer-readable media according to claim 15, wherein the matter oscillation includes matter vibration and/or molecular rotation.

17. One or more computer-readable media according to claim 10, wherein the facilitating of the medical diagnosis includes employing human-programmed heuristic rules and/or rules derived from machine learning operations.

18. A method comprising:
obtaining medical data about a particular internal organ or tissue of a living subject;
determining material composition of the particular internal organ or tissue of a living subject, the determining being based upon the obtained medical data;
generating a baseline model of the particular internal organ or tissue at the one point in time and a subsequent model of the particular internal organ or tissue at the later point in time, wherein each model is a three-dimensional data representation of the material composition of the particular internal organ or tissue at their respective time;
finding differences in material composition of the particular internal organ or tissue of the living subject by comparing and/or contrasting the baseline and subsequent models of the particular internal organ or tissue;
facilitating a medical diagnosis of the subject, the medical diagnosis being based, at least in part, upon a correlation between the differences in material composition of the particular internal organ or tissue and medical diagnostic data;
reporting the medical diagnosis of the subject.

19. A method according to claim 18, wherein the wherein the determining operation is based upon scans of the particular internal organ or tissue performed by a magnetic imaging resonance (MRI) machine.

20. A method according to claim 18, wherein:
the medical data includes information about matter oscillations within the particular organs or tissue of the living subject;
the determining operation being based upon one or more correlations between the matter oscillations and material composition.

21. A method according to claim 18, wherein:
the medical data includes information about matter oscillations within the particular organs or tissue of the living subject, wherein the matter oscillations within the particular internal organ or tissue is derived from one or more scans of the particular internal organ or tissue performed by a magnetic imaging resonance (MRI) machine;
the determining operation being based upon one or more correlations between the matter oscillations and material composition.
